(19) United States
(12) Patent Application Publication
(10) Pub. No.: US 2023/0031385 A1
(43) Pub. Date: Feb. 2, 2023

(54) STORAGE MEDIA FOR PRESERVATION OF CORNEAL TISSUE

(71) Applicant: National Health Research Institutes, Zhunan Township (TW)

(72) Inventors: Feng-Huei LIN, Zhunan Township (TW); Yu-Chun CHEN, Zhunan Township (TW); Wen-Yu SU, Zhunan Township (TW); Yen-Hsien LEE, New Taipei City (TW); Ko-Hua CHEN, Taipei (TW)

(21) Appl. No.: 17/380,474

(22) Filed: Jul. 20, 2021

(51) Int. Cl. A01N 1/02; C12N 5/079

(52) U.S. Cl. CPC A01N 1/0226; C12N 5/0621

(57) ABSTRACT

A corneal storage composition is disclosed. The composition comprises a buffered, balanced, nutrient and electrolyte aqueous solution; γ-PGA; and ferulic acid, in amounts effective to maintain cell integrity and viability of the corneal tissue for a period of greater than 14 days.

### FIELD OF THE INVENTION

The present invention relates generally to an ophthalmic solution, and more specifically to a storage medium for corneal tissue for subsequent transplant.

### BACKGROUND OF THE INVENTION

The cornea is the eye's outermost layer. It is a clear, dome-shaped surface that covers the front of the eye. Corneal tissue comprises five basic layers: the epithelial layer, Bowman's layer, the stroma, Decemet's membrane, and the endothelial layer. The cells of the endothelial layer play the most significant role in keeping the cornea clear. Under normal conditions, fluid travels slowly from the inside of the eye into the middle of the corneal layer (stroma). The endothelium's primary task is to pump this excess fluid out of the stroma. Without this pumping action, the stroma would swell with water, become hazy, and ultimately become opaque. Often due to pathology or an injury, endothelial cells may be destroyed. For all practical purposes, corneal endothelial cells do not reproduce. Thus, when endothelial cells are lost, either by trauma or disease, the lost cells are not replaced, and adjacent endothelial cells migrate and expand into the defect.

Corneal edema (swelling) occurs when the endothelial cell density is below a certain level, usually below about 400 cells/mm². Clear vision depends on corneal clarity, which in turn depends on corneal deturgescence. Advanced corneal swelling is associated with severe vision loss and blindness. Corneal opacities due to disease or scarring are also associated with visual loss and, if severe, blindness.

Corneal transplantation is the only available therapy to remedy irreversible corneal edema or scarring. Corneal transplant therapy involves replacing the diseased, scarred or traumatized cornea with a healthy, clear cornea obtained from an organ donor.

As in other cases of organ transplantation, it is necessary to perform various tests on the donor's blood to rule out various infectious diseases that may be transmitted to the recipient, resulting in a delay in the surgical procedure being performed. Not infrequently, donor tissue must be transported, causing further delay. Therefore, it is necessary to undertake in vitro storage or maintenance of the harvested tissue and cells until transplantation occurs. With a donor cornea, the corneal tissue must be preserved in a way that the viability and density of the corneal cells, particularly those of the endothelial layer, are preserved, and that the cornea does not swell and maintains its clarity.

Various solutions and media have been developed to preserve tissues for storage and/or transportation for later use. However, even with specially prepared available storage media, the practical storage life is limited. For example, the widely used McCarey-Kaufman medium is considered to be useful in the preservation of corneal tissues for up to four days. The most commonly used medium in U.S. eyebanks for corneal preservation is the commercially available Optisol™ solution (Bausch & Lomb), designed for intermediate storage at 4° C. Optisol™ solution is a serum-free medium containing a base medium, a buffer, chondroitin sulfate, dextran (molecular weight 40K), vitamins, and ATP precursors. Although studies, (e.g., Kaufman et al. Arch Ophthalmol; 109:864-868 (1991); Lindstrom et al. Am Journal of Ophthalmol; 114:345-346 (1992)) have reported that Optisol™ is effective at preserving endothelial structure for up to two weeks, there is a significant loss of endothelial viability after this time and the majority of surgeons prefer not to use a cornea which has been stored for more than about five days in this medium. There is therefore a need in the art to develop an improved corneal storage medium that facilitates the preservation of corneal tissue over a longer period of time.

Therefore, a heretofore unaddressed need exists in the art to address the aforementioned deficiencies and inadequacies related to corneal cell preservation, especially in connection with corneal storage for subsequent transplant.

### SUMMARY OF INVENTION

The present invention, a new formula of medium, comprise specific polymer to provide protections to the ocular tissues caused by an external osmolarity regulating. This well keeps the stromal hydration in order to maintain corneal transparency. Another ingredient is an abundant phenolic phytochemical found in plant cell wall components. It has antioxidant anti-bacterial properties and neuroprotection. So, this present invention can avoid swelling the donor cornea and provide good preserved conditions. The disclosed composition is particularly useful as an ophthalmic storage medium. It may be used in surgeries in general, although other uses, for example, tissue storage and preparation for grafting, and topical application are also contemplated. The present invention relates to a novel composition having a built-in mechanism that utilizes poly-γ-glutamic acid (γ-PGA) that is serve as an agent to adjust the osmolarity and avoided edema phenomena of tissue. It was used differential γ-PGA concentrations to adjust osmolarity in range of 330 to 390 mOsM which maintain the hydration within the corneal stroma during storage. It is ten times more hydrating than hyaluronic acid and far more elastic than collagen. Another ingredient is ferulic acid which had many pharmacological attributes including anti-inflammatory and antioxidant. This storage medium is more effective for protecting the cornea and retina during the mild or long-term storage, and furthermore it costs much less.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the osmosis pressure was γ-PGA concentration-dependent.

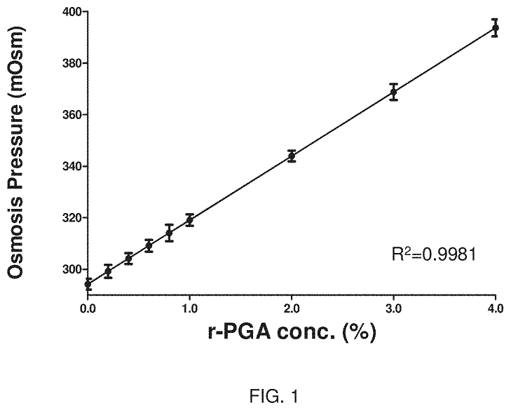

Osmosis pressures were measured by osmometer produced by Advanced Instrument, Co., Inc.

FIG. 2 shows γ-PGA had no cytotoxic effect on bovine corneal endothelium cells. CytoTox 96′ assay was used to evaluate the cytotoxicity according to ISO 10993.

FIG. 3 shows ferulic acid (50 μM and 100 μM) had no cytotoxic effect on human corneal endothelium cells (HCECs). The HCECs were assessed for apoptosis using flow cytometry staining with annexin-V conjugated with fluorescein isothiocyanate and propidium iodide.

FIG. 4 shows ferulic acid (50 μM and 100 μM) prevented and/or antagonized AAPH (25 mM) induced damage to bovine corneal endothelium cells. BCECs were assessed for apoptosis using a terminal deoxynucleotidyl transferase-mediated dUTP nick end-labeling (TUNEL) assay.

FIG. 5 shows a storage medium comprising γ-PGA and ferulic acid effectively reduced swelling of stroma. 5A and 5B: Tissue sections of rabbit cornea after storage at 4° C. for 14 days in a medium comprising γ-PGA and ferulic acid. 5C and 5D: Tissue sections of rabbit cornea after storage at 4° C. for 14 days in a cornea storage medium comprising M199 cell culture medium. 5A and 5C: HE staining. 5B and 5D: DAPI fluorescent staining of nucleus.

FIG. 6 shows a storage medium comprising γ-PGA and ferulic acid could keep the connections between corneal endothelium cells intact and maintained the nuclei in an even and complete shape. A-D: Tissue sections of rabbit cornea after storage at 4° C. for 7 days in a medium comprising γ-PGA and ferulic acid. A and D: ZO-1 staining. B and D: DAPI fluorescent staining of nucleus.

FIG. 7 shows storage media comprising γ-PGA and ferulic acid could maintain the corneal endothelium cell viability much better than Optisol™ media after storage at 4° C. for 14 days and 21 days, respectively.

### DETAILED DESCRIPTION OF THE INVENTION

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

A defined storage medium containing components which maintain and enhance the preservation of eve tissues at temperatures from about 4° C. to 37° C., the medium comprising effective amounts of deturgescent agent, antioxidant and anti-inflammation. The invention provides methods and compositions for preserving tissue, particularly corneal tissue for cold storage and transplantation. In one embodiment, the invention provides defined storage medium, a unique cornea preservation medium based on fundamental physiological principles and on research findings from our laboratory regarding maintenance of cell membrane integrity. The medium is typically provided in sterile solution. More detailed descriptions of the ingredients and their respective functions are described below.

The LCD storage medium of the invention differs from prior formulations in a number of specific ways. For example, the base medium used in the preferred formulation, OptiMEM (GIBCO/Life Technologies), contains ingredients important for supporting cell proliferation, such as insulin, transferrin, and selenium. Other nutrient media containing these three ingredients could substitute for OptiMEM. The growth medium of the invention also contains γ-PGA, ferulic acid, mannose 6-phosphate and thymosin β₄.

Cornea swelling may be prevented by the addition of water-retentive compounds to the preservation medium. Among these, one of the most used is the deturgescent compound, dextran, either alone (McKarey, B. B. and Kaufman, H. E. (1974) Invest. Ophthalmol. Vis. Sci. 13, 165) or in association with the glucosaminoglycan chondroitin sulfate (Kaufman H. E. et al. (1991) Arch. Ophthalmol. 109, 864-868). However, chondroitin sulfate is a heterogeneous compound because of the varied distribution of the sulfate molecules within the polymer (Scott, 1995). As a result, the compositions to be used for corneal storage may vary between lots. In addition, due to the sulfate molecules, chondroitin sulfate carries a strong negative charge. it has been reported that chondroitin sulfate can penetrate the cornea and favor its swelling, particularly upon rewarming the tissue from 4° C. to room temperature, before transplant (Kaufman et al. 1991). In an attempt to decrease the corneal swelling induced by chondroitin sulfate, cornea preservation compositions were formulated which contain deturgescent agents such as dextran, in combination with chondroitin sulfate (EP 0 517 972). However, dextran may penetrate the stroma during storage and may increase the swelling pressure on rewarming. In addition, it is now also clear that dextran can be toxic to the cornea, inducing senescence and degeneration (Chen et al. 1996). Yet, the poly-γ-glutamic acid) (γ-PGA) had a molecular weight of 10,000 up to 2 million. Such a high molecular weight γ-PGA is too viscous to be suitable as a storage medium component and must be used in combination with some other water-retaining component to prevent cornea swelling without augmenting the viscosity of the solution. It is, therefore, an object of the present invention to provide a cornea storage fluid capable of providing suitable storage conditions for viable cornea, while avoiding the drawbacks of prior fluids.

Oxygen-free radicals may injure the epithelium and endothelium of corneas in storage awaiting transplantation. Ferulic acid is a higher antioxidant activity then L-ascorbic acid and alpha-tocopherol. L-ascorbic acid (vitamin C) is one of the relatively few topical agents whose effectiveness against wrinkles and fine lines is backed by a fair amount of reliable scientific evidence. Unfortunately, vitamin C is relatively unstable. When exposed to air, vitamin C solution undergoes oxidation and becomes not only ineffective but also potentially harmful.

α-tocopherol (Vitamin E) is a fat-soluble antioxidant. In living systems, vitamins C and E can regenerate each other and thus potentiate each other's antioxidant effects. While the capacity of vitamin E to protect vitamin C from oxidation in a water solution is relatively modest, vitamin E enhances the antioxidant effects of vitamin C. Studies indicate that the combination of vitamins C and E provide better protection from UV-induced damage than either vitamin alone. On the other hand, vitamin E appears to have little effect on the ability of vitamin C to stimulate the synthesis of collagen.

Ferulic acid is a naturally occurring phenolic compound found primarily in plant cell walls. It is a potent antioxidant and may have skin benefits even when used alone. However, skin care related studies of ferulic acid focused on its ability to enhance the effects of vitamins C and E. Preliminary research indicates that ferulic acid may improve the stability of Vitamin C in water solution. Also, the addition of ferulic acid to the combination of vitamins C and E appears to increase protection from UV-induced skin damage. While the combination of vitamin C, E and ferulic acid appears to have clear advantages over vitamin C alone. Procysteine combination of ferulic acid (FA) and ascorbic acid and alpha-tocopherol may synergistic protective effect on lipid peroxidation.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

The γ-PGA, an anionic peptide, is a natural compound produced as capsular substance or as slime by members of the genus Bacillus (Crit. Rev. Biotechnol. 2001; 21:219-232). The γ-PGA is unique in that it is composed of naturally occurring L-glutamic acid linked together through amide bonds. It is reported from literature that this naturally occurring. γ-PGA is a water-soluble, biodegradable, and non-toxic polymer. Due to the unique properties on ion trapping and high water-absorbance, it has been widely used in various applications, such as metal chelate, absorbent cryoprotectant, ageing inhibitor, drug carrier or humectant. During the past few years, γ-PGA has been widely used as a biomaterial with fine swelling ability and biocompatibility makes it practicable for use in such clinical fields as bioglue, tissue engineering and drug delivery systems. The invention relates to the discovery that γ-PGA has the ability to adjust the osmolarity.

Ferulic acid is a higher antioxidant activity and light stably, to prevent corneal endothelium damage and cataract formation. Ferulic acid (FA), a polyphenol very abundant in vegetables and maize bran, is derivatives from the roots of Chinese Angelica, commonly name as “dong quai”. Several evidences have shown that ferulic acid acts as a potent antioxidant in vitro, due to its ability to scavenge free radicals and induce a robust cell stress response through the up-regulation of cytoprotective enzymes such as heme oxygenase-1, extracellular signal-regulated kinase 1/2 and Akt. Furthermore, ferulic acid inhibited the expression and/or activity of cytotoxic enzymes including inducible nitric oxide synthase, caspases and cyclooxygenase-2.

Procysteine is a cell permeable precursor of glutathione. Procysteine, a modified form of cysteine, is somewhat less toxic, and much more stable “on the shelf” than Cysteine. Procysteine maintains cellular levels of glutathione; amino acid supplies substrates for mitochondria. Procysteine improved recovery of rat hearts from ischemia/reperfusion injury. (The Journal of Nutritional Biochemistry Volume 5, Issue 7, July 1994,).

Thymosin β₄ sulfoxide is an anti-inflammatory agent generated by monocytes in the presence of glucocorticoids. (Nat Med. 1999 December; 5(12):1424-7.) Thymosin 134 (T134) is a water-soluble, 43-amino acid acidic polypeptide (pI 5.1) with a molecular weight of 4.9 kDa. T134 may exert its anti-inflammatory effects by regulating the activity of NFκB, a key modulator of inflammation. (Experimental Eye Research Volume 84, Issue 4, April 2007, Pages 663-669)

In further embodiments, the ophthalmic formulation may include Mannose 6-phosphate (M6P). M6P is a molecule bound by lectin in the immune system. It is theorized that M6P acts by competing with latent Transforming Growth Factor beta (TGFb) at the Insulin Like Growth Factor II receptor. (G. Sutton, et al., Mannose 6-phosphate reduces haze following excimer laser photorefractive keratectomy, Lasers and Light, Vol. 7, No. 2/3, pp. 117-119 (1996)) Mannose-6-phosphate significantly reduced TGF-beta1-mediated transformation of human corneal fibroblasts into myofibroblasts; it is a potential modulator of corneal wound healing and may reduce haze after refractive surgery. (J Cataract Refract Surg. 2010 January; 36(1):121-6.)

### Example 1: Formulation

The LCD medium composition comprised of poly-γ-glutamic acid is described in Table 1, in comparison with commercially available corneal storage media containing chondroitin sulfate and dextran (Optisol-GS) or dextran alone (McKarey-Kaufman).

TABLE 1
Characteristics of the LCD medium compared with those
of MK1 ™ and Optisol-GS ®

| | Components of Fluid | | |
|---|---|---|---|
| | MK1 ™ | Optisol-GS ™ | LCD |
| Medium | TC 199 | TC 199 MEM | MEM/DMEM |
| Buffer | HEPES 25 mM | HEPES 25 mM | HEPES 25 mM |
| | NaHCO₃ | | NaHCO₃ |
| | 0.35 g/L | | 0.35 g/L |
| pH | 7.2-7.4 | 7.2-7.4 | 7.1-7.5 |
| Osmolarity | 310-330 | 350-360 | 330-390 |
| | mOsm/kg | mOsm/kg | mOsm/kg |
| Antibiotic | Gentamycin | Gentamycin | Gentamycin |
| | | Streptomycin | Streptomycin |
| Poly-γ- | — | — | yes |
| glutamic acid | | | |
| Ferulic acid | — | — | yes |
| EGF | — | — | yes |
| Procysteine | — | — | yes |
| Thymosin β₄ | — | — | yes |
| D-Mannose | — | — | yes |
| 6-phosphate | | | |

OPTI-MEM I is a modification of Eagle's Minimal Essential Medium, buffered with HEPES and sodium bicarbonate, and supplemented with hypoxanthine, thymidine, sodium pyruvate, L-glutamine or GLUTAMAX, trace elements and growth factors. Phenol red is included at a reduced concentration as a pH indicator. The preferred formulation of the LCD medium of the invention has the following composition (Table 2).

TABLE 2
Formulation of the lcd medium

| Base Medium | MEM/DMEM | |
|---|---|---|
| HEPES | 25 | mM |
| NaHCO₃ | 0.35 | g/L |
| pH | 7.1-7.5 | |
| Osmolarity | 330-390 | mOsm/kg |
| Poly-γ-glutamic acid | 1-5% | |
| Ferulic acid | 0.1-5 | mM |
| Gentamycin | 10-500 | μg/ml |
| Streptomycin | 20-1000 | μg/ml |
| EGF | 5-1000 | ng/ml |
| Procysteine | 10-500 | μM |
| Thymosin β₄ | 10-500 | μM |
| D-Mannose 6-phosphate | 50-1000 | μM |

### Example 2: γ-PGA Exhibits No Significant Cytotoxicity on Corneal Endothelium Cells

The CytoTox 96® Assay quantitatively measures lactate dehydrogenase (LDH), a stable cytosolic enzyme that is released upon cell lysis. The CytoTox 96® Assay can be used to measure cell death following treatment with various concentration of γ-PGA. Bovine cornea endothelium cells were seeded on 96-well culture plate at a density of 1×10⁴ cells/well. Add various concentration of γ-PGA (from 0.2% to 4%) to appropriate wells so the final volume is 100-150 μl in each well and incubate cells at 37° C. for 1 and 7 days. The negative control is culture medium without γ-PGA. At each time point, transfer 50 μl aliquots from all test and control wells to a fresh 96-well flat clear bottom plate. And then, Add 50 μl of the CytoTox 96® Reagent to each sample aliquot. Cover the plate with foil or an opaque box to protect it from light and incubate for 30 minutes at room temperature. Add 50 μl of Stop Solution to each well of the 96-well plate. Pop any large bubbles using a syringe needle, and record the absorbance at 490 nm within 1 hour after adding the Stop Solution. Use the corrected values in the following formula to compute percent cytotoxicity:

$$Percent cytotoxicity = 100 \times \frac{Experimental LDH Release ( {OD}_{490} )}{Maximum LDH Release ( {OD}_{490} )}$$

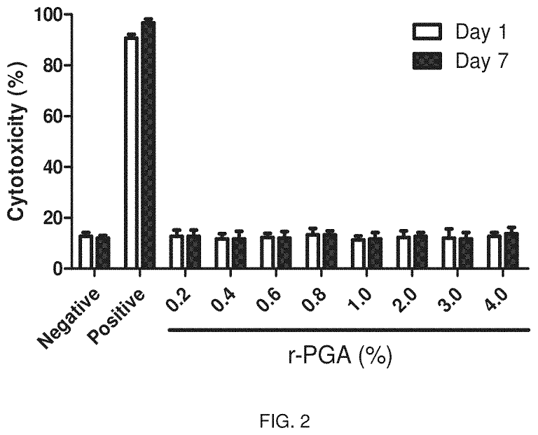

All γ-PGA groups had no cytotoxic effect on bovine corneal endothelium cells (FIG. 2).

### Example 3: Ferulic Acid Exhibits No Significant Cytotoxicity on Corneal Endothelium Cells

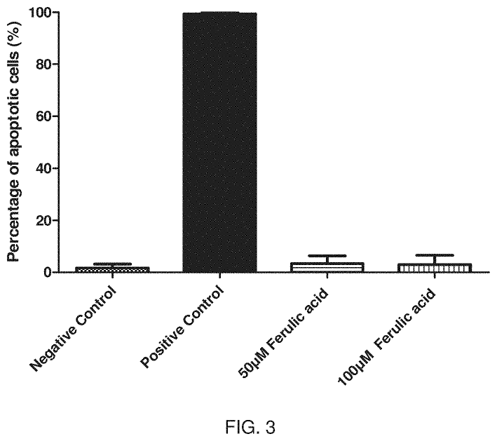

Annexin V staining is a common method for detecting apoptotic cells. The human corneal endothelium cells (HCECs) were assessed for apoptosis using flow cytometry staining with annexin-V conjugated with fluorescein isothiocyanate and propidium iodide (FIG. 3). HCECs were cultured in DMEM/F12 containing 50 μM and 100 μM ferulic acid. Briefly, HCECs were grown to confluence and incubated for 24 hours in 24-well plates using the same conditions as in the previous sets of experiments. After centrifugation and washing in cold PBS, HCECs for annexin V-FITC staining were resuspended in binding buffer (10 mM HEPES, 140 mM sodium chloride, 5 mM potassium chloride, 1 mM magnesium dichloride, 2.5 mM calcium dichloride, pH 7.4) at a concentration of 10⁶ cells/ml. Five hundred microliters containing 5×10⁵ cells was transferred to a culture tube, and 1.25 mL of FITC-conjugated annexin V was added. Positive controls were provided for both apoptotic and necrotic (10% ethanol) cell death. For simultaneous scoring of the differential cellular response, aliquots of 10⁴ cells each were immediately processed for fluorescence-activated cell sorting (FACS) on a FACS Calibur flow cytometer (Becton-Dickinson). Excitation parameters were set at λ_{Ex}=488 nm, and fluorescence emission was detected at 2Em=518 nm for annexin V-FITC.

### Example 4: Ferulic Acid Prevents AAPH Induced Apoptosis to Corneal Endothelium Cells

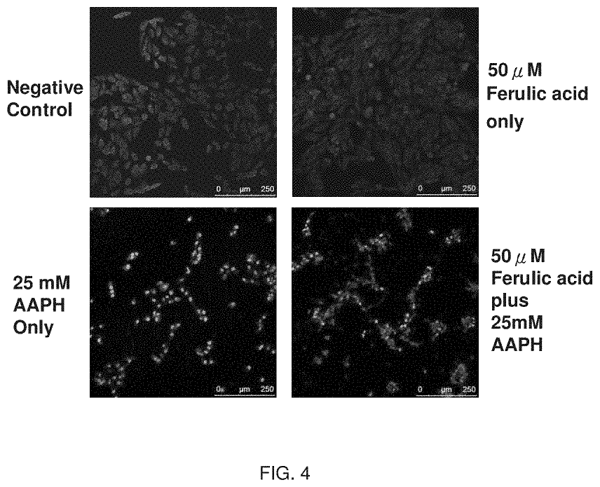

Terminal deoxynucleotidyl transferase (TdT) end-labeling (TUNEL) method (DeadEnd™ Fluorometric TUNEL System, Promega, Madison, USA) was used for apoptosis analysis according to the manufacturer's instructions (FIG. 4). Briefly, bovine cornea endothelium cells were seeded on LabTek® chamber slides at a density of 1×10⁴ cells/well for 24 hours, treated with 50 μM ferulic acid for 3 hours, and then reacted with 25 mM AAPH (2,2′-azobis(2-amidinopropane dihydrochloride) for 6 hours. Slides were immersed in 4% formaldehyde for fixation, cells were permeabilized with Triton® X-100. Then equilibration buffer, nucleotide mix and rTdT enzyme were added in for apoptosis cells labeling. Finally, counterstaining the nucleus with propidium iodide, and observed by fluorescence microscopy. The green fluorescence spots in FIG. 4 represents the apoptotic cells (fluorescein-12-dUTP) with a red background (propidium iodide).

### Example 5: The Storage Medium Comprising γ-PGA and Ferulic Acid Effectively Reduced Swelling of Stroma

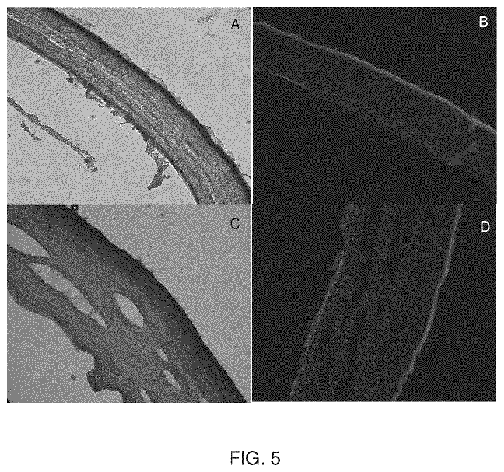

Enucleated rabbit eyes with intact eyelids that protect the corneal surface were obtained from New-Zealand rabbits, placed in Hank's balanced salt solution (pH 7.4). After surgical removal of the eyelids and ocular muscles, the rabbit cornea was preserved at 4° C. for 14 days in storage medium comprising γ-PGA and ferulic acid and M199 medium, respectively. Afterwards, the cornea tissues were immediately fixed in 4% formaldehyde, and embedded in paraffin. 4 mm sections were stained with H&E, and viewed under a light microscope. Additionally, the specimens were mounted with 40,6-diamidino-2-phenylindole (DAPI) nuclear staining and examined under a fluorescence microscope (FIG. 5). The storage medium comprising γ-PGA and ferulic acid effectively reduced swelling of stroma.

### Example 6: The Storage Medium Comprising γ-PGA and Ferulic Acid could Keep the Connections Between Corneal Endothelium Cells Intact and Maintained the Nuclei in an Even and Complete Shape

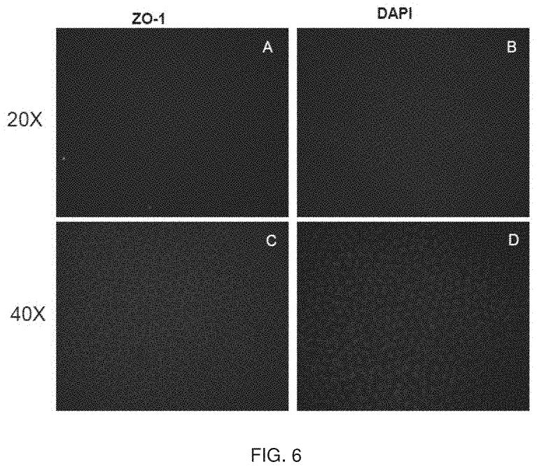

For immunostaining of the whole mount rabbit corneas, the tissue were enucleated and preserved at storage medium comprising γ-PGA and ferulic acid at 4° C. for 7 days. Afterward, the corneas were excised and incubated in 20 mM EDTA for 30 minutes at 37° C. and blocked in PBS/0.2% TritonX-100/1% BSA for one hour at room temperature. Anti-ZO-1 (1:400; Sigma) antibodies were prepared in the blocking solution and incubated with the rabbit corneas overnight at 4° C. The corneas were then washed with PBS, incubated with the appropriate secondary antibody for 3 hours at room temperature, and counterstained with DAPI. FIG. 6 demonstrates a storage medium comprising γ-PGA and ferulic acid could keep the connections between corneal endothelium cells intact and maintained the nuclei in an even and complete shape.

### Example 7: The Storage Media Comprising γ-PGA and Ferulic Acid could Maintain the Corneal Endothelium Cell Viability

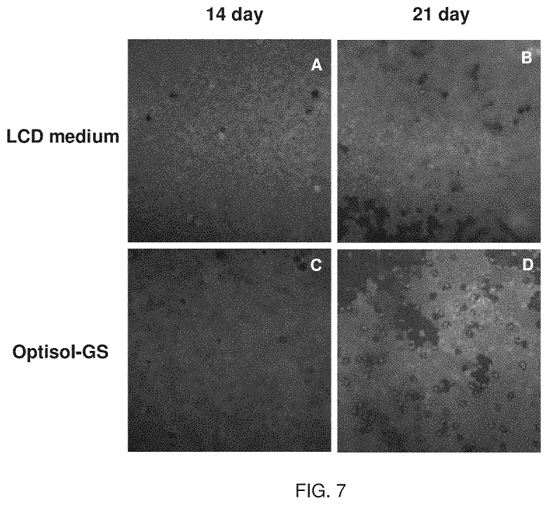

The Live/Dead staining kit utilizes two fluorescent dyes, calcein-AM and ethidium homodimer (EthD-1). Calcein AM (a non-fluorescent molecule) can be hydrolyzed by intracellular esterases into the highly negatively charged green fluorescent calcein in live cells. EthD-1 is a high-affinity nucleic acid stain that is weakly fluorescent until bound to DNA, yielding a bright red fluorescence in dead cells. The rabbit cornea were preserved in LCD medium (The storage medium comprising γ-PGA and ferulic acid) and Optisol-GS at 4° C. for 14 days and 21 days, respectively. Afterward, the medium was removed and washed by PBS for 3 times. And then, The Live/Dead staining regent were added on the cornea endothelium layer for 3 min and observed by fluorescent microscope immediately (FIG. 7).

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A corneal storage composition comprising:
(a) A buffered, balanced, nutrient and electrolyte aqueous solution,
(b) γ-PGA, and
(c) ferulic acid.

2. The corneal storage composition of claim 1, wherein the concentration of γ-PGA ranges from 1 to 5%.

3. The corneal storage composition of claim 1, wherein the concentration of ferulic acid ranges from 0.1 to 5 mM.

4. The corneal storage composition of claim 1, further comprising at least one ingredient chosen from EGF, procystein, thymosin β₄ and D-mannose 6-phosphate.

5. The corneal storage composition of claim 4, wherein the concentrations of EGF, procystein, thymosin β₄ and D-mannose 6-phosphate range from 5 to 1000 ng/ml, 10 to 500 μM, 10 to 500 μM, and 50 to 1000 μM, respectively.

6. The corneal storage composition of claim 1, further comprising EGF, procystein, thymosin β₄ and D-mannose 6-phosphate.

7. The corneal storage composition of claim 1, wherein the composition is free of dextran and chondroitin sulfate.

8. The corneal storage composition of claim 1, wherein the composition is free of glucose.

9. The corneal storage composition of claim 1, wherein the composition is free of ß-mercaptoethanol.

10. A method for storage and preservation of a corneal tissue comprising: placing the corneal tissue in a corneal storage composition of claim 1.

11. The method of claim 10, wherein the cell viability of the corneal tissue is maintained for a period of at least 14 days.

12. The method of claim 10, wherein the corneal storage composition is maintained at a temperature between 2 to 8° C.

13. The method of claim 12, wherein the corneal storage composition is maintained at a temperature of 4° C.

14. A method for reducing swelling of a corneal tissue during storage, comprising:
storing the corneal tissue in a corneal storage composition of claim 1.
